(19) United States
(12) Plant Patent Application Publication
(10) Pub. No.: US 2025/0081867 P1
(43) Pub. Date: Mar. 6, 2025

(54) Apple Tree Named 'PremA133'

(50) ;

(71) Applicant: Prevar Limited, Hastings (NZ)

(72) Inventor: Richard Volz, Havelock North (NZ)

(73) Assignee: Prevar Limited, Hastings (NZ)

(21) Appl. No.: 18/798,378

(22) Filed: Aug. 8, 2024

(30) Foreign Application Priority Data
Sep. 5, 2023 (FR) 21414

(51) Int. Cl. A01H 6/74

(52) U.S. Cl. CPC A01H 6/7418
USPC PLT/161

(57) ABSTRACT

A new and distinct apple tree named ‘PremA133’ notable for its fruit, which has an attractive red skin color, appealing flavor, and large size, and for its double resistance to apple scab (Venturia inaequalis).

Latin name: Malus domestica.

Variety denomination: PremA133.

### BACKGROUND OF THE VARIETY

The new apple tree ‘PremA133’ was created in the course of a planned breeding program carried out at Havelock North, New Zealand. ‘PremA133’ originated from a controlled cross of ‘A180R20T066’ (seed parent, not patented) and ‘A185R09T179’ (pollen parent, not patented) performed at Havelock North, New Zealand in October 2008. ‘PremA133’ was selected from a population of seedlings derived from the parents, and was singled out for further observation for its fruit, which has an attractive red skin color and appealing flavor. The variety was first asexually propagated by grafting onto ‘M9’ rootstock (not patented) at Havelock North, New Zealand in August 2015. ‘PremA133’ trees have been observed over a number of asexually propagated generations, and have been found to remain true to type.

‘PremA133’ is distinguished similar varieties as set forth in Table 1 below.

TABLE 1
Comparison of ‘PremA133’ to Similar Varieties

| Comparison Variety | Characteristic | In Comparison Variety | In ‘PremA133’ |
|---|---|---|---|
| ‘PremA134’ | Lenticel Prominence | Low | High |
| (not patented) | | | |
| ‘PremA129’ | Red Skin Color | Red | Pink-Red |
| (USPP 29,214) | | | |
| ‘Sciros’ | Maturity Time | Early to mid- | Early October |
| (USPP 7,814) | | September | |
| ‘Cripps Pink’ | Maturity Time | Late October to | Early October |
| (USPP 7,880) | | early November | |

### BRIEF DESCRIPTION OF THE PHOTOGRAPHS

The attached photographs of trees and fruit were taken at Havelock, New Zealand during the 2023/2024 growing season. The trees depicted were planted in 2018 and grown on ‘M9’ rootstock (not patented).

FIG. 1 shows the fruit of a ‘PremA133’ apple tree;

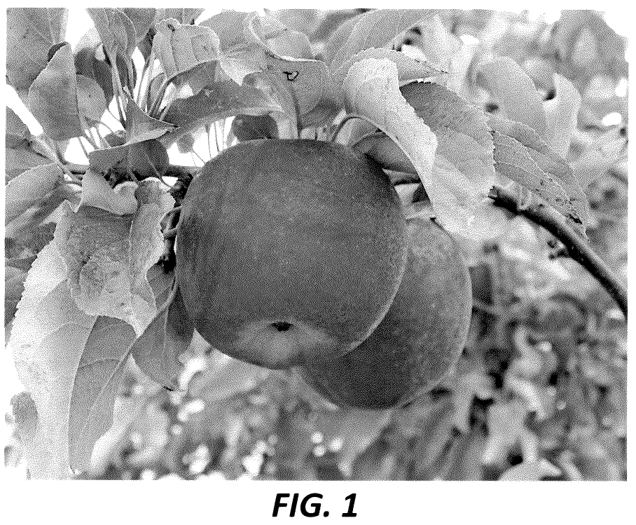

FIG. 2 shows the flower buds of a ‘PremA133’ apple tree;

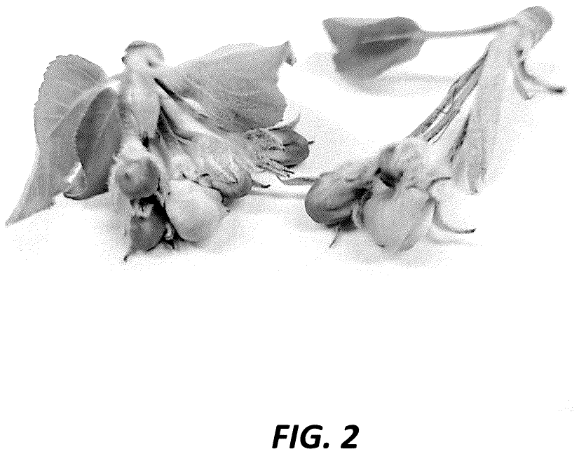

FIG. 3 shows the flowers of a ‘PremA133’ apple tree;

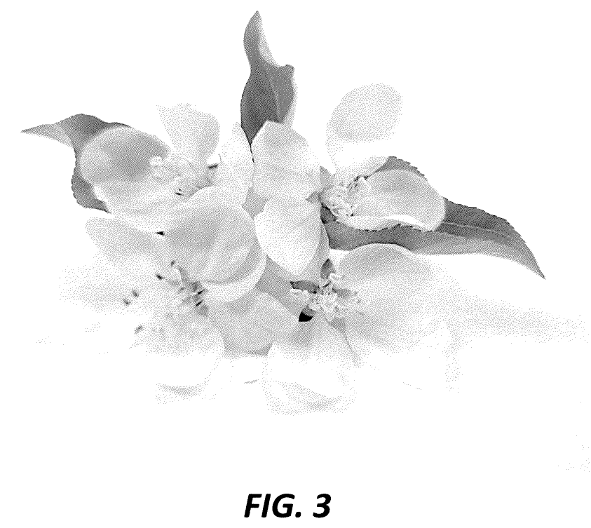

FIG. 4 shows ‘PremA133’ apple trees in bloom;

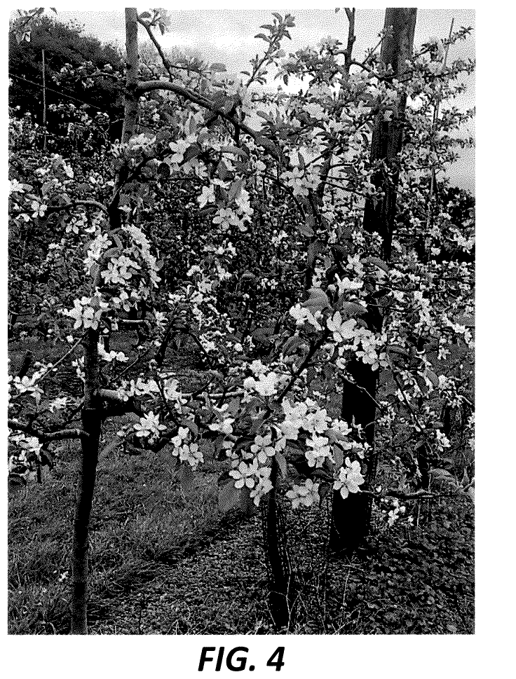

FIG. 5 shows the upper surface of leaves of a ‘PremA133’ apple tree;

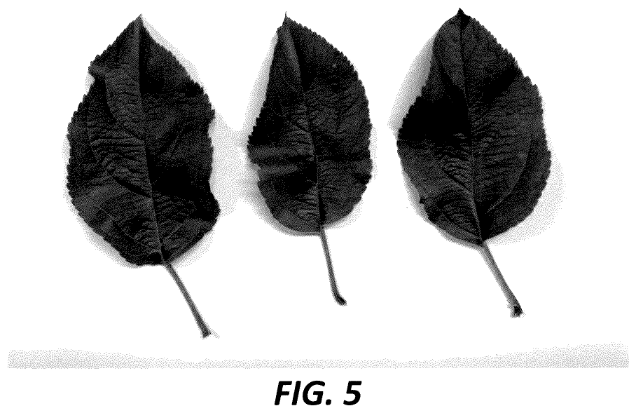

FIG. 6 shows the lower surface of leaves of a ‘PremA133’ apple tree; and,

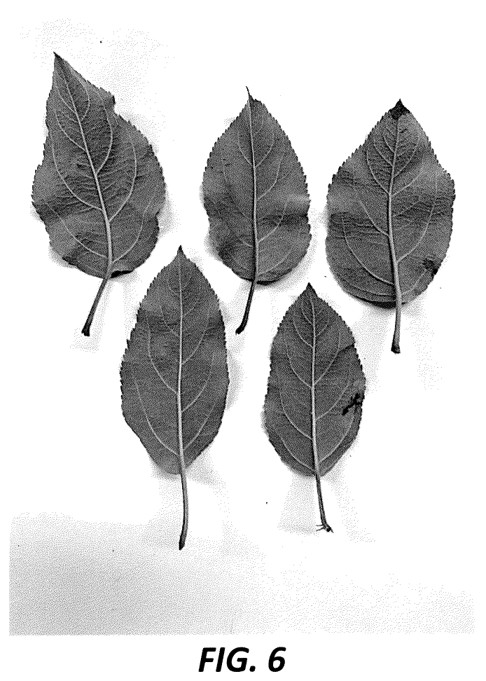

FIG. 7 shows a ‘PremA133’ apple tree and its fruit.

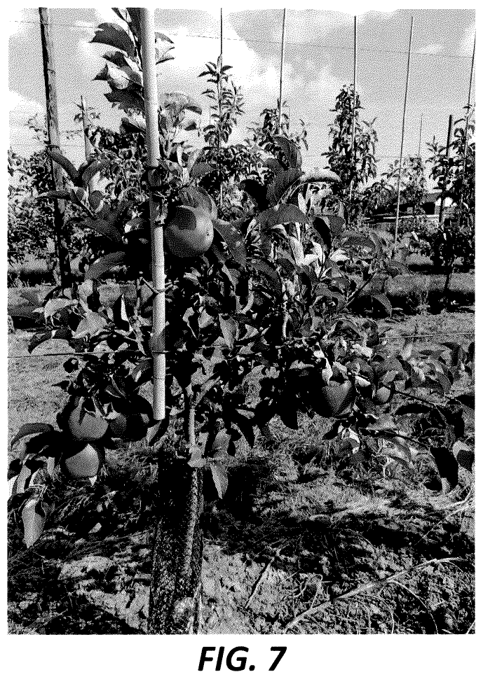

### DETAILED BOTANICAL DESCRIPTION OF THE VARIETY

The following detailed botanical description is based on trees and fruit observed at Havelock, New Zealand during the 2023/2024 growing season. The described trees were planted in 2018 and grown on ‘M9’ rootstock (not patented). It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average. All colors are described with reference to The R.H.S. Colour Chart, 6^{th} ed. (Royal Horticultural Society, 2015).
Tree:
   Vigor.—Medium.
   Type.—Ramified
   Habit.—Spreading.
   Height.—1.8 m.
   Spread.—3.2 m.
   Trunk diameter (at 30 cm above the graft).—12.7 cm.
   Bearing.—On spurs.
   Bark texture.—Smooth to medium with raised lenticels.
   Bark color.—Greyed-green 197B; with pubescence removed, grey-brown 199B.
   Lenticel length.—1.4 mm to 1.7 mm.
   Lenticel width.—0.4 mm to 0.6 mm.
   Lenticel shape.—Elongated.
   Lenticel color.—Greyed-white 156C.
   Lenticel density.—45 per 10 cm².
Branch (fruiting branches located at around 1 m above the graft union):
   Length.—89 cm.
   Diameter.—20 mm.
   Crotch angle.—75° to 85° (trained).
   Bark color.—Grey brown N199D (pubescence removed).
   Branch lenticel length.—0.9 mm to 1.2 mm.
   Branch lenticel width.—0.4 mm to 0.6 mm.
   Branch lenticel shape.—Elongated.
   Branch lenticel color.—Brown N200B.
   Branch lenticel density.—5 to 7 per cm².
One year old shoot:
   Length.—48 cm.
   Diameter.—9 mm.
   Color.—Grey-brown N199C.
   Pubescence.—Sparse, moderate.
   Shoot lenticel length.—0.8 mm to 1.1 mm.
   Shoot lenticel width.—<0.5 mm.
   Shoot lenticel shape.—Round to elongated.
   Shoot lenticel density.—5 per cm².
Flower buds:
   Stage described.—First pink.
   Quantity per spur.—Average 5.
   Bud shape.—Obovate.
   Apex shape.—Rounded.
   Length.—8.4 mm.
   Diameter.—5.0 mm.
   Color.—Red 39B.
Flowers:
   Diameter of fully open flower.—41 mm.
   Depth of fully open flower.—10 mm.
   Relative position of petal margins.—Free.
   Quantity per cluster.—Average 5.
   Date of first bloom.—Oct. 12 (2023).
   Date of full bloom.—Oct. 21 (2023).
   Pollination requirement.—‘Fuji’ (not patented) and other late flowering varieties.
Petals:
   Quantity per flower.—5.
   Petal shape.—Obovate.
   Length.—17 mm.
   Width.—12 mm.
   Apex shape.—Obtuse.
   Base shape.—Acute.
   Margin.—Weak undulation; curled.
   Color of upper surface.—White NN155D.
   Color of lower surface.—White NN155B with minimal weak veins red-purple 76C.
Pitsils:
   Length.—9.7 mm.
   Color.—Yellow-green N144A.
Stigma:
   Diameter.—<0.5 mm.
   Color.—Yellow-green 151A.
Style:
   Length.—7.6 mm.
   Color.—Yellow-green N144A.
Stamens:
   Quantity.—Average 20.
   Length.—7.3 mm.
   Anther color.—Yellow-green 153D.
   Pollen.—Moderate.
Pedicel:
   Length.—23.8 mm.
   Diameter.—1.4 mm.
   Color.—Greyed orange 174A with pubescence.
Sepals:
   Quantity.—5.
   Color.—Yellow-green 150A.
   Shape.—Acute triangular.
   Apex shape.—Acute.
   Margin.—Smooth, reflexed.
Leaves:
   Shape.—Ovate.
   Length.—10 cm.
   Width.—5.7 cm.
   Blade margin.—Serrate.
   Apex.—Acuminate.
   Base shape.—Rounded.
   Color of upper surface.—Green N137B.
   Color of lower surface.—Yellow-green 144A.
   Pubescence.—Moderately present on lower surface.
   Attitude in relation to shoot.—Upward.
Petiole:
   Length.—28.7 mm.
   Diameter.—1.8 mm.
   Color.—Yellow-green N144C with moderate to high anthocyanin coloration purple N79C.
Fruit:
   Quantity per cluster.—2 to 3 (after thinning).
   Diameter.—77 mm.
   Height.—66 mm.
   Weight.—204 g.
   General shape in profile.—Globose.
   Position of maximum diameter.—Near equator.
   Ribbing.—Weak.
   Crowning at calyx end.—Weak.
   Size of eye.—Small.
   Aperture of eye.—Closed.
   Depth of eye basin.—10 mm.
   Width of eye basin.—32.4 mm.
   Length of sepal.—4 mm to 4 mm.
   Bloom of skin.—Present, moderate.
   Greasiness of skin.—Absent or weak.
   Background color of skin.—Yellow-green 154C.
   Amount of overcolor.—Large to very large, 90%.
   Overcolor of skin.—Red 53C.
   Intensity of over color.—Moderate to Intense.
   Pattern of over color.—Solid flush.
   Number of lenticels.—Few to medium.
   Size of lenticels.—Medium to large.
   Amount of russet around stalk cavity.—Absent.
   Amount of russet on cheeks.—Absent or weak.
   Amount of russet around eye basin.—Absent or weak.
   Length of stalk.—24.3 mm.
   Thickness of stalk.—2.3 mm.
   Depth of stalk cavity.—17.6 mm.
   Width of stalk cavity.—34.5 mm.
   Stalk color.—Grey-brown N199D.
   Firmness of flesh.—Firm, 8.15 kgf/cm².
   Flesh texture.—Fine to medium.
   Juiciness.—Moderate.
   Aroma.—Moderate.
   Brix.—13.8° Brix.
   Flesh color.—Yellow-white 158C.
Seeds:
   Quantity per fruit.—5 to 7.
   Shape.—Ovate.
   Length.—9 mm to 10 mm.
   Width.—4 mm to 6 mm.
   Color.—Greyed-orange 166C.
Harvest:
   Time for harvest.—Mar. 12 (2024, Havelock North, New Zealand); after ‘PremA096’ (U.S. Plant Pat. No. 24,946), before ‘Scilate’ (U.S. Plant Pat. No. 20,477).
   Number of picks.—One.
   Yield per tree.—80+ fruits.
   Disease and pest resistance.—Double apple scab (Venturia inaequalis) resistance (carries Rvi2 and Rvi6 genes).
   Storage characteristics.—6 months in 1-MCP storage.
   Market use.—Fresh.

I claim:

1. A new and distinct apple tree named ‘PremA133’, substantially as described and illustrated herein.
